(19) United States
(12) Patent Application Publication
(10) Pub. No.: US 2024/0325682 A1
(43) Pub. Date: Oct. 3, 2024

(54) METHOD BY WHICH ACTUATING CABLES ARE FIXED BY WELDING TO A COMPONENT IN PARTICULAR OF THE DISTAL HEAD OF A MEDICAL DEVICE

(71) Applicant: AXESS VISION TECHNOLOGY, JOUE-LES-TOURS (FR)

(72) Inventors: Emmanuel HALLAUER, SACHE (FR); Philippe LE ROUX, TOURS (FR); Jérôme COURATIN, SAINT-PIERRE-DE-CORPS (FR)

(21) Appl. No.: 18/578,727

(22) PCT Filed: Jul. 12, 2022
(86) PCT No.: PCT/FR2022/051403

§ 371 Date: Jan. 12, 2024

(30) Foreign Application Priority Data
Jul. 13, 2021 (FR) FR21 07587

(51) Int. Cl. A61M 25/00; A61B 1/00; A61B 1/005; A61M 25/01

(52) U.S. Cl. CPC A61M 25/0009; A61B 1/0011; A61B 1/0052; A61M 25/0147

(57) ABSTRACT

A method for fixing one end of an actuation cable includes providing a part provided with at least one region made of thermoplastic material for fixing one end of an actuation cable and providing a serrated welding head formed of teeth alternately separated by hollows. The end of an actuation cable is then positioned on a region of the part made of thermoplastic material, heated, and the serrated welding head is applied with pressure onto the end of the actuation cable to ensure the penetration of the teeth into the region made of thermoplastic material to move the material in the hollows in order to obtain at least one bead of thermoplastic material trapping the actuation cable.

### TECHNICAL FIELD

The present disclosure relates to the technical field of fixing by welding, on a part made of thermoplastic material, of actuation cables forming a portion of a control mechanism ensuring a translational or rotational movement.

An object of the present disclosure finds a particularly advantageous application but not exclusively, for the devices allowing access to the interior of a body such as a cavity or a channel for example and it more specifically targets the medical devices of the catheter type and preferably, the medical devices of the endoscope type.

The medical device of the catheter or endoscope type according to the present disclosure is used for diagnostic, therapeutic or surgical purposes for the inspection of all internal parts of the human body accessible by natural or artificial means. For example, the medical device according to the present disclosure can be used in the field of the urinary tract, the gastrointestinal tract, the respiratory system, the cardiovascular system, the trachea, the sinus cavity, women's reproductive system, the abdominal cavity or any other part of the human body to be explored by a natural or artificial path.

### BACKGROUND

Generally, a medical endoscope includes, as described for example by patent application WO 2016/188537, a control handle to which an insertion tube is fixed. This tube includes a distal head equipped with an optical visualization system making it possible to illuminate and examine the organ, cavity or duct of the human body. Upstream of this distal head, the insertion tube includes a bending structure or deflection portion allowing the orientation of the distal head using one or several actuation cables mounted inside the insertion tube. Each actuation cable includes a distal end fixed to the distal head and a proximal end on which a control mechanism fitted to the handle acts to ensure the sliding of the cables and consequently, the folding of this deflection portion in order to orient the distal head.

Conventionally, this control mechanism includes a control lever acting on a pivoting part on which the proximal end of the actuation cables is fixed. The fixing of the actuation cables on the pivoting part is ensured by threading the end of each actuation cable in holes arranged in the pivoting part and by forming a loop. A tubular sleeve threaded onto the actuation cable is thread onto the actuation cable to allow it to stop in translation. Different fixing techniques are known, such as screwing. Generally, the operation of fixing the proximal end of the actuation cables on the pivoting part is a relatively delicate operation and likely to lead to an unreliable fixing and/or to an anchoring unlikely to allow correct operation.

In the state of the art, it is also known, from patent FR 2 477 465, a method for fixing a plate made of plastic material to the end of a hollow cylinder, also made of plastic material to the end of a cylinder also made of plastic material. For this purpose, a portion of the hollow cylinder is heated, folded inwards to be brought into contact with the plate, the whole then being heated and subjected to pressure in order to be assembled.

It is also known from patent application JP H10181466, a method for welding electrical cables on a thermoplastic part having grooves in each of which an electrical cable is engaged. As illustrated in FIG. 5, the side walls of the ribs are deformed using a welding head including a central cavity delimited on either side by teeth to move the thermoplastic material.

### SUMMARY

The present disclosure aims to overcome the drawbacks of the state of the art by proposing a new technique for fixing by welding, on a part made of thermoplastic material, one end of the actuation cables that is part of a control mechanism, this new technique eases implementation and reduces cost while presenting optimal fixing quality that is sustainable over time.

To achieve such an objective, the object of the present disclosure relates to a method aimed at fixing on a part, the end of an actuation cable that is part of a control mechanism, the method including the following steps:
providing a part being part of a control mechanism, this part being provided with at least one region made of thermoplastic material for fixing one end of an actuation cable;
providing a serrated welding head formed of teeth alternately separated by hollows;
positioning the end of an actuation cable on a region made of thermoplastic material of the part;
applying heat to the region of the part and applying the serrated welding head with a pressure on the end of the actuation cable to ensure the penetration of the teeth into the region made of thermoplastic material to move the material into the hollows in order to obtain at least one bead of thermoplastic material trapping the actuation cable with a view to fixing, by welding, the actuation cable with the part.

Advantageously, a tension of the actuation cable is applied when the pressure is exerted on the end of the actuation cable by the serrated welding head.

Advantageously, a serrated welding head, including a number of hollows comprised between 1 and 15 and preferably between 5 and 8 to move the material with a view to obtaining a corresponding number of consecutive beads of thermoplastic material, is provided.

For example, a serrated welding head, including a series of hollows having a depth comprised between 0.15 mm and 6 mm to move the material in order to obtain beads of determined height, is provided.

According to another characteristic of the present disclosure, a serrated welding head, including a series of hollows having a determined width to move the material in order to obtain beads of width projecting from either side of the actuation cable, is provided.

Advantageously, a serrated welding head, including a series of teeth each having a triangular shape to move the material in order to obtain beads of corresponding shape, is provided.

Preferably, a portion of the actuation cable is positioned on the region made of thermoplastic material, using positioning structures arranged on said region.

Advantageously, an ultrasonic welding ensures the fixing of the actuation cable to the part.

According to another example of implementation, a hot and pressure riveting ensures the fixing of the actuation cable to the part.

According to one preferred application, a pivoting part being part of a control mechanism making it possible to orient the distal head of a medical device is provided as a part, this pivoting part being provided with two regions made of thermoplastic material each for the fixing of a proximal end of an actuation cable.

Advantageously, the part of a control mechanism provided with at least one fixing region made of thermoplastic material has at least one bead made of thermoplastic material trapping an anchoring portion of an actuation cable embedded into the part by being located recessed from the portions of the actuation cable located on either side of the anchoring portion.

The part provided with two regions made of thermoplastic material each has a series of beads made of thermoplastic material each trapping an anchoring portion of an actuation cable embedded into the part by being located recessed from the portions of the actuation cable located on either side of the anchoring portion.

The part includes beads made of thermoplastic material trapping the actuation cable and by being located in the vicinity of the inlet of a guide groove for the actuation cable.

Advantageously, the part includes beads in the form of successive serrations covering the actuation cable and separated from each other by recesses with which the actuation cable is flush.

According to one advantageous embodiment characteristic, the part is a pivoting part and is part of a control mechanism making it possible to orient the distal head of a medical device.

Preferably, the part is in the form of a disk or of an annular ring to form an actuation pulley.

The object of the present disclosure advantageously aims a control handle for a medical device including a control mechanism making it possible to orient the distal head of the medical device, the control mechanism including a part in accordance with the present disclosure.

Various other characteristics appear from the description given below with reference to the appended drawings which show, by way of non-limiting examples, embodiments of the object of the present disclosure.

### BRIEF DESCRIPTION OF THE DRAWINGS

FIG. 1 is a schematic view of a medical device of the catheter or endoscope type in the general sense including a control handle provided with a control mechanism making it possible to orient the distal head of the medical device.

FIG. 2 is a perspective view showing a pivoting part made in the form of a pulley provided with two actuation cables.

FIG. 3 is a perspective view showing a pivoting part made in the form of a pulley on which two actuation cables are intended to be fixed.

FIG. 4 is a perspective view showing one exemplary embodiment of a welding head intended to fix an actuation cable on a pivoting part.

FIG. 5 is a schematic view showing, before fixing by a welding head, the positioning of an actuation cable on a part.

FIG. 6 is a schematic view showing, after fixing by a welding head, the anchoring of an actuation cable on a part.

FIG. 7 is a schematic view in elevation section, showing after fixing by a welding head, the anchoring of an actuation cable on a part.

FIG. 8 is a schematic top view, showing after fixing by a welding head, the anchoring of an actuation cable on a part.

FIG. 9 is a schematic top view, showing after fixing by a welding head, another exemplary embodiment of the anchoring of an actuation cable on a part.

### DETAILED DESCRIPTION

An object of the present disclosure relates to a new technique for fixing on a part, the end of actuation cables that are part of a control mechanism that ensures a translational or rotational movement. The object of the present disclosure finds applications in numerous technical fields such as, for example, machines, vehicles or medical devices.

The present disclosure finds a particularly advantageous application for fixing the actuation cables of a bending structure that makes it possible to orient the distal head of medical devices of the catheter or endoscope type. The following description illustrates, by way of example, the implementation of the present disclosure, for a medical device 1 of the endoscope or catheter type in the general sense designed to access the interior of a body such as a cavity or a channel for example.

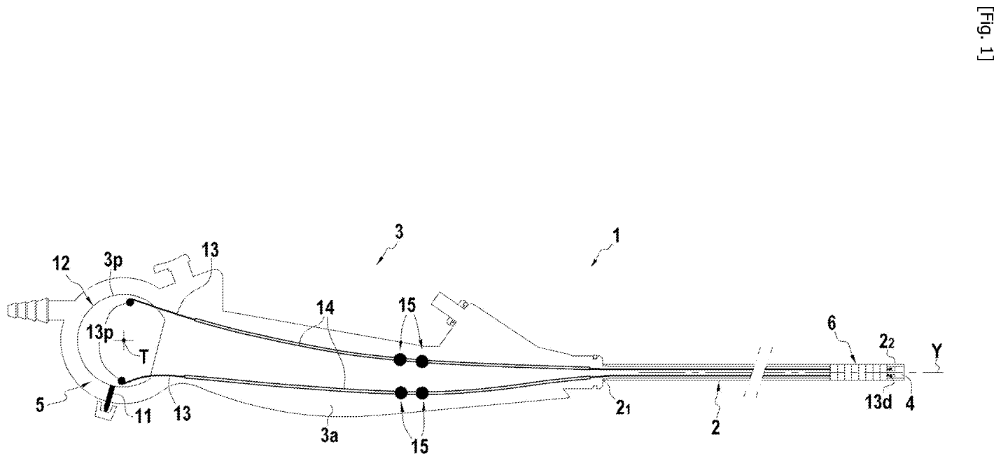

As can be seen more specifically in FIG. 1, a medical device 1 of the endoscope or catheter type includes an insertion tube 2 having on one side, a proximal portion 2₁ connected to the casing 3a of a control handle 3 and on the opposite side, a distal portion 2₂, which is equipped with a distal head 4. The insertion tube 2 is fixed temporarily or permanently on the casing of the control handle 3. This insertion tube 2, which has a greater or lesser length and flexibility is intended to be introduced into a natural or artificial access means with a view to carrying out various operations or functions for therapeutic, surgical or diagnostic purposes. The insertion tube 2 is made of a semi-rigid material and has a length adapted to the length of the duct to be inspected and which can be comprised between 5 cm and 2 m. The insertion tube 2 has various cross-sectional shapes such as a square, an oval or a circular shape. This insertion tube 2, which is in contact with the tissues, human organs or medical equipment (trocars or probes), is essentially for single or multiple use by a patient or even for reusable use after decontamination, disinfection or sterilization.

According to one preferred exemplary embodiment, the medical device 1 in accordance with the present disclosure is an endoscope including a vision system able to illuminate and bring back an image of the distal portion of the insertion tube 2. The endoscope thus includes a vision system mounted inside the control handle 3 and penetrating inside the insertion tube 2 up to the distal head 4.

Conventionally, the medical device 1 also includes a control mechanism 5 making it possible to orient the distal head 4 relative to the longitudinal axis Y of the insertion tube 2. For this purpose, the insertion tube 2 includes upstream of the distal head 4, a bending, folding or deflection portion 6 allowing the orientation of the distal head 4 relative to the longitudinal axis Y of the insertion tube 2. This bending, folding or deflection portion 6 can be made in any appropriate manner to ensure the bending of the distal head 4 relative to the longitudinal axis Y of the insertion tube 2. For example, this bending, folding or deflection portion 6 can be made by a spring or by tubular vertebrae articulated together.

The control mechanism 5 can be made in any suitable manner so that the distal head 4 can be moved between a rest position in which the insertion tube 2 is rectilinear and a deflectable position in which the deflection portion 6 is curved. By way of non-limiting example, the control mechanism 5 can correspond to the control mechanism described in patent FR 3 047 887. For this purpose, the control mechanism 5 includes a manual control lever 11 accessible from the outside of the casing 3a of the control handle. This control lever 11 acts directly or indirectly on at least one fixing or support part 12 to cause the rotation of the part about a transverse axis of rotation T. This part 12 is connected to the distal head 4 so that a rotation of the part 12 causes the bending of the distal head 4.

Thus, following the application of a manual effort on the control lever 11, the bending of the distal head 4 is obtained. According to one exemplary embodiment illustrated in FIG. 1, the control lever 11 is a lever guided in rotation over a limited angular region, along a transverse axis of rotation T. It should be noted that the control lever can be made in a different way such as in the form of a pusher guided in linear displacement and biased in elastic return to act on the part via a system for transforming the linear movement of the pusher into a rotational movement of the part 12.

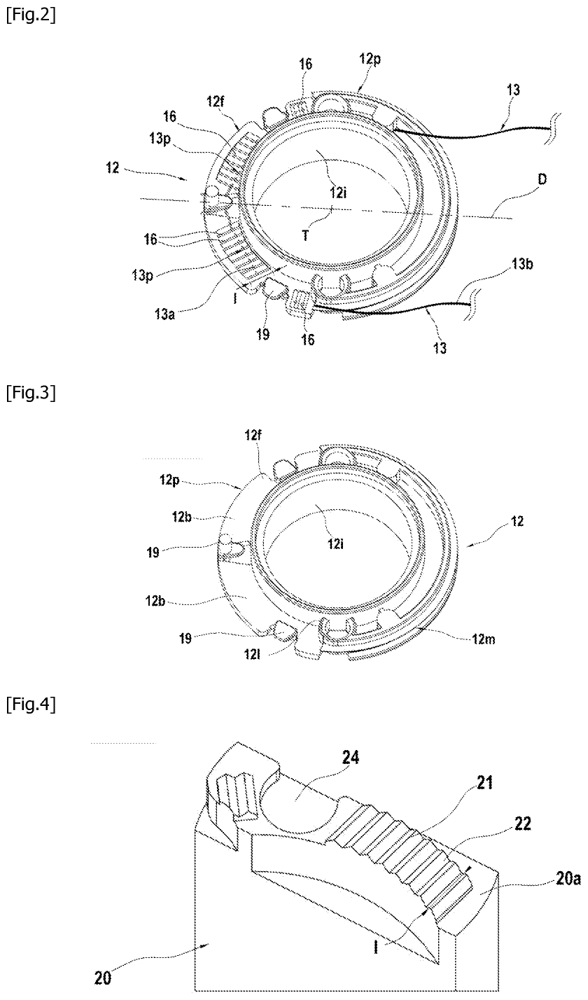

This part 12 is in the exemplary embodiment, guided in rotation by an annular bearing 3p arranged in the casing or by a part added into the casing 3a. The part 12 is guided by a pivot connection so as to present only a rotational movement about the transverse axis of rotation T. The part 12 can be made in different ways by considering that the rotational movement of the part is limited to less than one revolution and particularly, less than one third of a revolution. According to the preferred exemplary embodiment illustrated in FIGS. 2 and 3, the part 12 is a pivoting part made in the form of a flat annular ring delimited by a circular peripheral edge 12p and a circular interior edge 12i. This part 12 thus includes two planar or flat annular faces 12f extending on either side of the part. This part 12 forms a pulley as will be better understood later in the description. Of course, the part 12 can be made in a different way such as in the form of a semi-ring or an annulus segment for example. Likewise, the part 12 can be made in the form of a solid disk or a cam to vary the bending in a non-linear manner.

FIGS. 2 and 3 show one exemplary embodiment of the part in the form of an annular ring 12 which is not provided with the control lever 11. For example, the control lever 11 is able to be added onto a planar face 12f by any appropriate means such as by interlocking of a stud into a housing 12l of the annular ring. Of course, the control lever 11 and the annular ring 12 can also be manufactured in one piece.

At least one and in the example illustrated, two actuation cables 13 are fixed to the part 12. These actuation cables 13 are mounted inside the insertion tube 2 to be fixed to the distal head 4. The proximal ends 13p of the actuation cables 13 are fixed to the part 12 while the distal ends 13d of the actuation cables 13 are fixed to the distal head 4. In the example illustrated, the proximal ends 13p of the actuation cables 13 are fixed in a symmetrically opposite manner on the part 12 relative to a diametral plane.

This control mechanism 5 is adapted to ensure for example left-right or up-down displacement of the distal head 4. Of course, the control mechanism 5 can be adapted to ensure the left-right and up-down displacement of the distal head 4 using three or four actuation cables 13. In the example illustrated, the control lever 11 has a rotational stroke but as already described, it is possible that the control lever 11 has a translation movement causing the rotation of the part 12 via a movement transformation system.

Of course, the actuation cables 13 are made in any appropriate manner to ensure the movement transformation function leading to the bending of the distal head 4. Thus, these cables 13 can be made for example by rods, yarns, braids, filaments, strands or warps, made of a metal or polymer material for example. Typically, the actuation cables 13 are made of stainless steel braids with a diameter comprised between 0.05 mm and 4 mm.

Each actuation cable 13 is mounted inside a support sheath 14 intended to be fixed to the casing 3a of the control handle 3, using a fixing system 15 of all types known per se. The support sheath 14 thus ensures the slidable guiding of the actuation cable 13 during the operations of deflection the distal head 4. The actuation cable 13 thus moves in translation relative to the support sheath 14 which is mounted secured to the control handle 3.

In accordance with the present disclosure, the fixing of the actuation cables 13 with the part 12 is performed by welding. More specifically, the actuation cables 13 are fixed in the example illustrated, by their proximal portion 13p on either or both of the planar faces 12f of the part 12. For this purpose, it is considered that the part 12 presents through its planar faces 12f, for each actuation cable 13, a fixing region 12b which is planar in the illustrated example. The two fixing regions 12b are located in a symmetrically opposite manner relative to a diametral plane D of the part 12 passing through the transverse axis of rotation T. Preferably, the two fixing regions 12b are located outside the diametral plane D to allow the actuation cables 13 to be guided over a significant angular region of the part as will be explained in the remainder of the description. Of course, it can be envisaged that each planar face 12f of the part 12 includes a fixing region 12b.

In accordance with the present disclosure, the part 12 includes for each actuation cable 13, a fixing region 12b having, after the welding operation, at least one bead 16 and preferably a series of beads 16 trapping an actuation cable 13 anchored to the part 12. Each bead 16 is an excrescence or a protuberance of material made by the welding operation and coming from the material constituting the part 12. Each bead 16 protruding relative to this fixing region 12b by rising above the actuation cable 13. Advantageously, the beads 16 are arranged consecutively along a portion of the actuation cable 13. The proximal end 13p of each actuation cable 13 is fixed by welding, via the beads 16 in a safe and efficient manner so that the movement of the part 12 can be transmitted to the distal head 4.

In the example illustrated in the Figures, the control mechanism 5 includes two actuation cables 13 each fixed to the part 12 using the fixing beads 16 in accordance with the present disclosure. Of course, only one actuation cable 13 can be fixed to the part 12.

As appearing more specifically in FIGS. 2 and 7, the proximal end 13p of each actuation cable 13 thus includes an anchoring portion 13a trapped by the beads 16 and being prolonged on one side, by an actuation portion 13b which ends with the distal end 13d fixed to the distal head 4. Preferably, the anchoring portion 13a of the actuation cable 13 is prolonged opposite to its actuation portion 13b, by an end portion or ending 13c.

According to one advantageous variant of embodiment, the part 12 is arranged to allow the positioning and guiding of the actuation cable 13. Thus, the part 12 includes a circular groove 12m centered on the axis of rotation T of the part and arranged in the planar face 12f on the periphery of the part 12. During the pivoting of the part 12, each actuation cable 13 is thus guided by the circular groove 12m.

Advantageously, the circular groove 12m is arranged with a depth adapted to house the actuation cables 13 which are kept in contact with the part due to the tension imposed on the actuation cables 13 when the part 12 is mounted in the casing 3a of the control handle. This part 12 provided with the actuation cables forms an actuation pulley able to form a portion of the actuation mechanism 5 for an endoscope in the example described.

According to one advantageous embodiment characteristic, the part 12 includes beads 16 made of thermoplastic material trapping the actuation cable 13 by being located in the vicinity of the inlet of the guide groove 12m for the actuation cable. Such beads 16 allow the correct positioning of the actuation cables in the guide grooves 12m. As is apparent more specifically from FIG. 2, the part 12 includes two series of consecutive beads 16 taking into account the configuration of the part but it is clear that the beads 16 can be arranged on the part in one or several series.

After the fixing of the actuation cable 13 to the part 12, the beads 16 are as it appears more specifically in FIGS. 2 and 7, in the form of successive serrations covering the actuation cable 13 and separated from each other by recesses 17 with which the actuation cable 13 is flush. The actuation cable 13 is embedded into the part 12 at the level of the serrations. In other words, the anchoring portion 13a of the actuation cable 13 is embedded into the part 12 in the sense that the anchoring portion 13a is located recessed from the actuation portion 13b and from the end portion or ending 13c. As illustrated in FIG. 7, the actuation portion 13b and the end portion or ending 13c extend in a plane passing through the fixing region 12b and from which the anchoring portion 13a of the actuation cable 13 is recessed. For example, the anchoring portion 13a of the actuation cable 13 extends substantially in a plane located recessed from the plane containing the actuation portion 13b and the end portion or ending 13c.

Advantageously, the beads 16 have a width I projecting from either side of the actuation cable 13, as is apparent more specifically from FIG. 2. Thus, the width of the beads 16 taken along a direction perpendicular to the direction of extension of the extension cable 13 is greater than the diameter of the actuation cable 13 to be able to project from each side of the actuation cable.

The beads 16 cooperate with the actuation cable 13 to connect the actuation cable 13 to the part 12. In other words, the beads 16 are in intimate contact with the actuation cable 13 while being able to be inserted into the thickness of the actuation cable from its external surface. The beads 16 in combination ensure the blocking in translation of the actuation cable 13.

According to one characteristic of the present disclosure, the actuation cable 13 is fixed by welding to the part 12. According to one characteristic of the present disclosure, the beads 16 are made of thermoplastic material that is to say of a material capable of being softened by heating above a certain temperature and hardened by cooling. For example, the material from which the beads 16 are made falls under the category of semi-crystalline or amorphous thermoplastic polymers. Typically, the beads 16 are for example made of ABS (acrylonitrile butadiene styrene), PP (polyprolylene), POM (polyoxymethylene), polyamide, polyurethane, PE (polyethylene), PS (polystyrene), or PA (Polyamide), PE (Polyethylene, SAN (Poly (styrene/acrylonitrile), PEEK (Polyetheretherketone), PPS (Polyphenylene sulfide), or any mixture of these polymers.

As the beads 16 are formed from the material constituting the fixing region 12b, the fixing region 12b of the part 12 is made of a thermoplastic material. According to one preferred variant of embodiment, the part 12 and the fixing region are made from the same thermoplastic material. Typically, the part 12 is manufactured according to the molding technique during which the part 12 is formed by being provided with the fixing region 12b. Of course, it could be envisaged to add the fixing region 12b made of thermoplastic material onto a part 12 made of all types of materials.

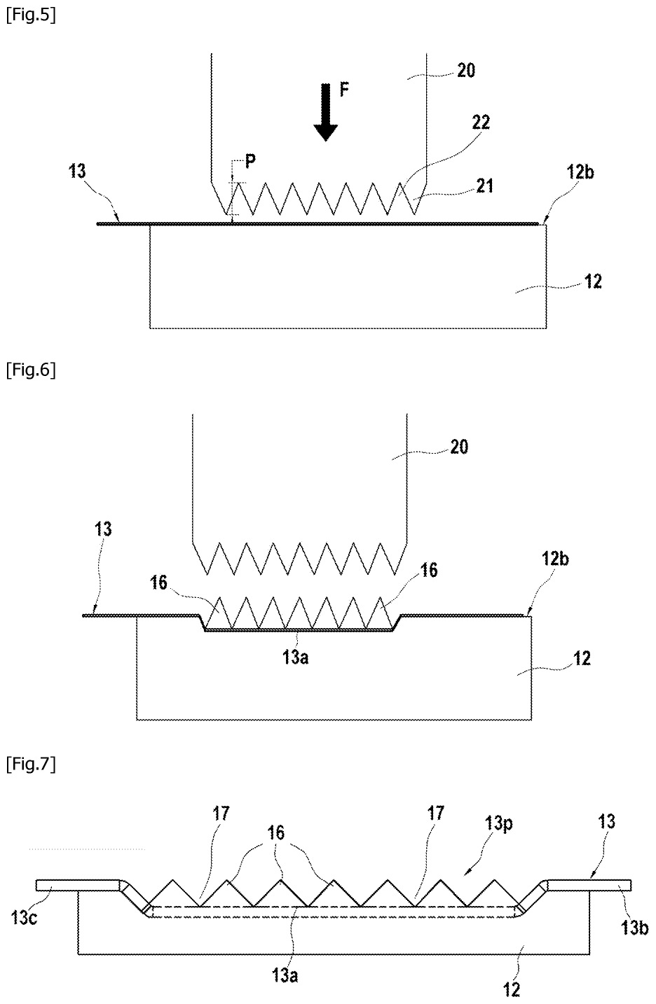

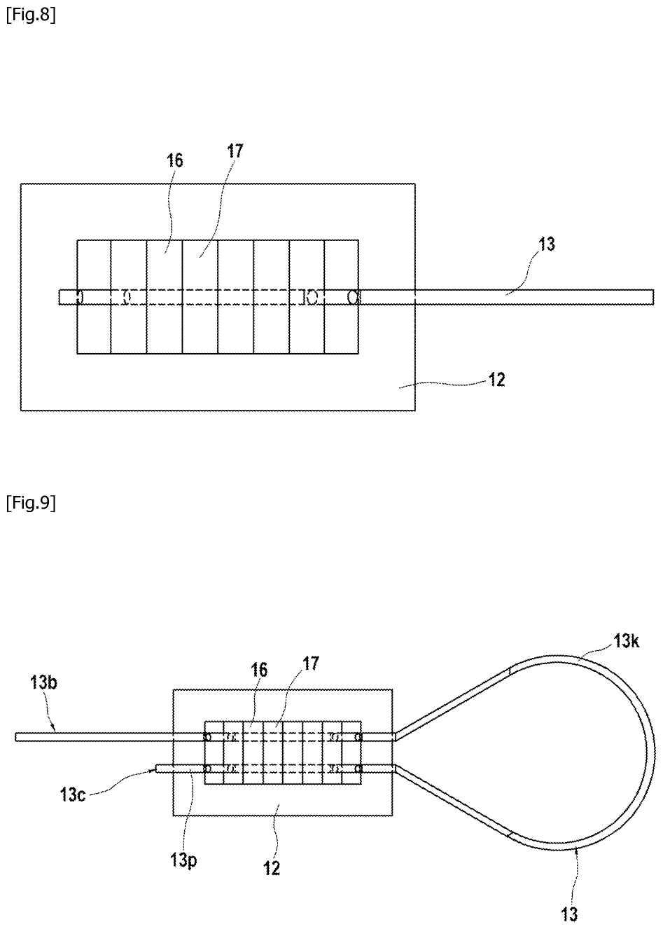

The fixing of an actuation cable 13 on the part 12 is carried out by the method described below in relation more specifically to FIGS. 5 and 6.

The first step of the fixing method consists in providing a part 12 for a control mechanism 5, provided with at least one region 12b made of thermoplastic material for the fixing of the proximal end 13p of the actuation cable 13. In the example illustrated in FIG. 2, the part 12 includes for the two actuation cables 13, two fixing regions 12b of planar surface.

According to one advantageous embodiment characteristic, the part 12 is provided with one or several structures 19 ensuring the positioning of the actuation cable 13 on the fixing region 12b. These positioning aid structures 19 can be in any form suitable for positioning and holding the actuation cable prior to the welding operation per se. As illustrated more specifically in FIG. 3, the positioning structures 19 can be in the form of studs or lugs.

The fixing method thus consists in positioning the proximal end 13p of each actuation cable 13 on a fixing region 12b made of thermoplastic material of the part 12. The step of placing the actuation cable 13 on the part can be carried out manually or automatically using a robotic arm.

The fixing method also consists in providing a serrated welding head 20 formed of teeth 21 alternately separated by hollows 22. It should be understood that the number of teeth 21 and hollows 22 is chosen according to the number of beads 16 desired. Indeed, as will be explained in detail in the remainder of the description, each bead 16 is formed by the material present in a trough 22 located between two consecutive teeth 21. The serrated welding head 20 includes a number of hollows 22 comprised between 1 and 15, preferably between 3 and 12 and, according to one advantageous variant of embodiment, between 5 and 8.

As appears more particularly from FIGS. 4 and 5, the teeth 21 and the hollows 22 are arranged at the terminal portion of the serrated welding head 20. The serrated welding head 20 has a transverse end face 20a from which the teeth 21 protrude. In the exemplary embodiment illustrated in the drawings, the serrated welding head 20 has a recess 24 leaving on one side, a first series of teeth 21 and hollows 22 and the other side, a second series of teeth 21 and hollows 22. The recess 24 is adapted to make a positioning structure 19.

According to one advantageous embodiment characteristic, the serrated welding head 20 includes a series of teeth 21 having a determined shape to move the material in order to obtain beads 16 of corresponding shape. The teeth 21 can take any shape suitable for making beads 16 of material. According to one advantageous embodiment characteristic illustrated in the drawings, the teeth 21 have a triangular shape. Each tooth 21 thus has a triangular transverse cross section. As illustrated in the drawings, the teeth 21 have, perpendicularly to their transverse cross section, a straight shape that is to say the teeth have rectilinear ridges over their entire width.

According to one advantageous embodiment characteristic, the serrated welding head 20 includes a series of hollows 22 having a depth p comprised between 0.15 mm and 6 mm to move the material in order to obtain beads 16 of determined height. As illustrated in FIG. 5, the depth p of a trough 22 corresponds to the distance taken along a direction perpendicular to the transverse end face 20a, between the bottom of the trough 22 and the end of a neighboring tooth 21.

According to another advantageous embodiment characteristic, the serrated welding head 20 includes a series of hollows 22 and teeth 21 having a width I determined to obtain beads 16 of width projecting from either side of the actuation cable. The width I of the hollows 22 and of the teeth 21 which is taken perpendicularly to the transverse cross section of the teeth 21 is comprised between 0.2 and 12 mm.

The fixing method consists of applying heat to the fixing region 12b and applying the serrated welding head 20 with a pressure on the proximal end 13p of the actuation cable 13 to ensure the penetration of the teeth 21 into the region made of thermoplastic material. The penetration of the serrated welding head 20 makes it possible to move, using the teeth 21, the material in the hollows 22 in order to obtain a series of consecutive beads 16 of thermoplastic material trapping the actuation cable 13 in order to fix it, by welding, with the part 12. The level of penetration of the serrated welding head 20 into the part 12 determines the volume of material moved intended to penetrate into the hollows 22. Depending on the volume of material moved, the beads 16 take more or less the form of the hollows 22. In the example illustrated in the drawings, the beads 16 have a triangular transverse cross section.

As shown in FIG. 5, at least the pressure force is exerted using a serrated welding head 20 adapted to the welding method implemented. Indeed, the heat can be provided by the serrated welding head 20 and/or by a device independent of the serrated welding head 20. In the same sense, the heat can be provided before the application of the pressure force or simultaneously with the application of the pressure force. This step of welding the actuation cable 13 can be carried out manually or automatically using a robotic arm. For example, the parameters of displacement of the serrated welding head 20 as well as the temperature parameters which can be defined to obtain efficient welding are able to be reproduced.

According to one advantageous implementation characteristic, a tension of the actuation cable 13 is applied when the pressure is exerted on the proximal end 13p of the actuation cable 13 by the serrated welding head 20. The tension on the actuation cable is exercised by all appropriate systems. This pre-stress tension applied on the actuation cable prevents clearances and ensures a responsive and functional deflection.

The heat input at the level of the free end of the fixing region 12b leads to its softening so that the material is moved under the effect of the application of a pressure along a direction of approximation of the serrated welding head 20 in the direction of the actuation cable 13, represented by the arrow F in FIG. 5. The direction of displacement of the serrated welding head 20 is advantageously established perpendicularly to the plane of extension of the fixing region 12b. Advantageously, the serrated welding head 20 is moved relative to the part 12. Of course, it can be envisaged to move the part 12 relative to the serrated welding head 20.

The fixing of the actuation cable 13 to the part 12 is made by the implementation of a hot snap-riveting method (namely laser, induction, hot air, infrared for example) or a friction (orbital, longitudinal or axial) vibration welding method.

According to one preferred exemplary implementation, the fixing of the actuation cable 3 to the part 12 is carried out by the implementation of an ultrasonic welding method. According to this exemplary embodiment, the part 12 is for example positioned on an anvil and the serrated welding head 20 such that a vibrating sonotrode is applied on the proximal end 13p of the actuation cable 13 supported by the fixing region 12b by being moved a few mm towards the inside of the fixing region to move the material from this portion of the part 12.

The method according to the present disclosure makes it possible to obtain a part 12 provided with beads 16 ensuring the fixing of actuation cables 13 of the distal head 4 of a medical device 1 of the endoscope or catheter type. This part 12 forms a portion of a control mechanism 5 mounted in a control handle for a medical device 1 of the endoscope or catheter type.

According to the preceding description, the object of the present disclosure relates to the fixing of the actuation cables 13 of the bending structure making it possible to orient the distal head of medical devices of the catheter or endoscope type. In the exemplary embodiment illustrated, the actuation cables 13 are fixed on the pulley forming a portion of the control mechanism. It should be noted that the actuation cable 13 can be fixed to a sleeve made of thermoplastic material intended to be mounted for example on the pulley forming a portion of the control mechanism.

In the preceding description, the actuation cable 13 is fixed to a part 12 by its proximal end 13p. It is clear that the actuation cable 13 can be fixed to a part 12 by its distal end or by any other of its portions.

FIG. 9 illustrates one variant of embodiment of the fixing of an actuation cable 13 on a part 12 made of thermoplastic material aimed at making a loop 13k at the end of the actuation cable 13. For this purpose, the actuation cable 13 is positioned on the part 12 to form outside the part 12, a loop 13k intended for example to surround a cylindrical part 12. The proximal end 13p of the actuation cable 13 is folded to be positioned in the vicinity of a segment of the actuation cable 13, for example parallel to each other. In this example, this segment of the actuation cable 13 and the proximal end 13p of the actuation cable 13 are fixed by a series of common beads 16. In other words, the fixing of the cable is ensured using a serrated welding head whose teeth 21 width ensures the simultaneous anchoring of the two segments of the actuation cable 13. Of course, each segment of the actuation cable 13 can be fixed using distinct beads 16.

It appears from the preceding description that the object of the present disclosure is adapted to ensure the fixing by welding, on a part 12 made of thermoplastic material, of actuation cables 13 forming a portion of a control mechanism of a medical device in the general sense. The object of the present disclosure finds other applications for ensuring the fixing by welding, on a part 12 made of thermoplastic material, of actuation cables 13 producing a rotational or translational movement. This is the case for example of the fixing of the actuation cables 13 with parts 12 forming a portion of the brake mechanism of a bicycle or of an acceleration mechanism of a vehicle.

1. A method for fixing one end of an actuation cable forming a portion of a control mechanism, the method including the following steps:
providing a part being part of a control mechanism, this part being provided with at least one region made of thermoplastic material for fixing one end of an actuation cable;
providing a serrated welding head formed of teeth alternately separated by hollows;
positioning the end of an actuation cable on a region made of thermoplastic material of the part;
applying heat to the region of the part; and
applying the serrated welding head with a pressure on the end of the actuation cable to ensure the penetration of the teeth into the region made of thermoplastic material to move the heated material into the hollows in order to obtain at least one bead of thermoplastic material trapping the actuation cable with a view to fixing, by welding, the actuation cable with the part.

2. The fixing method according to claim 1, wherein a tension of the actuation cable is applied when the pressure is exerted on the end of the actuation cable by the serrated welding head.

3. The fixing method according to claim 1, wherein a serrated welding head, including a number of hollows comprised between 1 and 15 and preferably between 5 and 8 to move the material with a view to obtaining a corresponding number of consecutive beads of thermoplastic material, is provided.

4. The fixing method according to claim 1, wherein a serrated welding head, including a series of hollows having a depth comprised between 0.15 mm and 6 mm to move the material in order to obtain beads of determined height, is provided.

5. The fixing method according to claim 1, wherein a serrated welding head, including a series of hollows having a determined width to move the material in order to obtain beads of width projecting from either side of the actuation cable, is provided.

6. The fixing method according to claim 1, wherein a serrated welding head including a series of teeth each having a triangular shape to move the material in order to obtain beads of corresponding shape is provided.

7. The fixing method according to claim 1, wherein a portion of the actuation cable is positioned on the region made of thermoplastic material, using positioning structures arranged on said region.

8. The fixing method according to claim 1, wherein an ultrasonic welding ensures the fixing of the actuation cable to the part.

9. The fixing method according to claim 1, wherein hot and pressure riveting ensures the fixing of the actuation cable to the part.

10. The fixing method according to claim 1, wherein a pivoting part being part of a control mechanism making it possible to orient the distal head of a medical device is provided as a part, this pivoting part being provided with two regions made of thermoplastic material each for the fixing of a proximal end of an actuation cable.

11. A part of a control mechanism provided with at least one fixing region made of thermoplastic material having at least one bead made of thermoplastic material trapping an anchoring portion of an actuation cable embedded into the part by being located recessed from the portions of the actuation cable located on either side of the anchoring portion.

12. The part according to claim 11, wherein it is provided with two regions made of thermoplastic material each having at least one bead made of thermoplastic material each trapping an anchoring portion of an actuation cable embedded into the part by being located recessed from the portions of the actuation cable located on either side of the anchoring portion.

13. The part according to claim 11, wherein it includes beads made of thermoplastic material trapping the actuation cable and by being located in the vicinity of the inlet of a guide groove for the actuation cable.

14. The part according to claim 11, wherein the beads are in the form of successive serrations covering the actuation cable and separated from each other by recesses with which the actuation cable is flush.

15. The part according to claim 11, wherein the part is a pivoting part being a part of a control mechanism making it possible to orient the distal head of a medical device.

16. The part according to claim 15, wherein it is in the form of a disk or of an annular ring to form an actuation pulley.

17. A control handle for a medical device including a control mechanism making it possible to orient the distal head of a medical device, the control mechanism including a part in accordance with claim 11.
